(19) United States
(12) Patent Application Publication
(10) Pub. No.: US 2013/0178552 A1
(43) Pub. Date: Jul. 11, 2013

(54) FILLERS FOR DENTAL COMPOSITES

(71) Applicants: Dirk Kruber, Alfter (DE); Thomas Doege, Eschweiler (DE)

(72) Inventors: Dirk Kruber, Alfter (DE); Thomas Doege, Eschweiler (DE)

(73) Assignee: Quarzwerke GmbH, Frechen (DE)

(21) Appl. No.: 13/809,759

(22) PCT Filed: Jul. 12, 2011
(86) PCT No.: PCT/EP2011/061793

§ 371 Date: Mar. 22, 2013

(30) Foreign Application Priority Data
Jul. 14, 2010 (EP) 10169498.2

(51) Int. Cl. A61K 6/083

(52) U.S. Cl. CPC A61K 6/083
USPC 522/71; 523/116

(57) ABSTRACT

A powdery filler for dental materials consisting of particles of feldspar or feldspar derivatives having a mean particle diameter (d50) of from 0.25 to 5 μm, said particles having a coating with a silicon compound containing reactive groups.

The present invention relates to fillers for dental materials.

In the dental field, composite materials have replaced traditional materials, such as amalgam. One of the essential reasons for this is improved aesthetics. Composite materials can be colored in a wide variety of colors, so that they match the color of the teeth.

Composite materials consist of a polymerizable synthetic resin and a filler. Typically, the polymerizable resin is cured with UV light. Therefore, it is necessary for the materials to be UV transparent. In many cases, the curable synthetic resins are acrylates, for example, bisphenol A-glycidyl methacrylate.

Typical fillers that are employed in composite materials today include silicas, glasses and ceramics. In composite materials, the fillers are contained in an amount of typically about 70 to 85%, so that they substantially codetermine the properties of the composite material. Properties of the filler material that particularly determine the properties of the composite material are the particle distribution and the particle shape.

In many cases, the filler is itself radiopaque, in order that the filler material can be recognized as a sharply outlined shape when X-ray images are made. However, there are also applications where radiopacity is not relevant.

In virtually all composite materials, it is necessary to pretreat the filler in order to achieve a strengthening of the binding between the filler material and synthetic resin. An essential aspect of the quality of a composite is the aspect of shrinking. A composite that shrinks opens a gap between the composite and the tooth, which may lead to further attack at the dental material.

Further properties relevant to practical application include good polishing properties, good handling properties, optical properties (e.g., UV transparency, low discoloring), good curing properties, and of course also the price.

Document U.S. Pat. No. 7,294,392 B2 discloses a composite material that is sintered from feldspar particles having a mean particle diameter d50 of 4.5 μm to form a porous matrix. The porous matrix thus formed is silanized in a subsequent step and filled with a polymer in a further step.

EP 1 225 867 B1 discloses a dental material made of silanized feldspar particles having a mean particle diameter of ≦0.3 μm.

From EP 0 747 034 A1, paste opaques are known that include feldspar particles having a mean particle diameter d50 of from 3 to 6 μm, among others.

Document U.S. Pat. No. 3,400,097 discloses frits made of silanized feldspar particles with particle sizes of from 200 to 325 mesh for the preparation of porcelain prostheses.

Although a wide variety of different composite materials and fillers for composite materials exist, there is still a need for further fillers having different properties, which preferably are improved at least in some areas.

It is the object of the present invention to provide such fillers.

This object is achieved by a powdery filler for dental materials consisting of particles of feldspar or feldspar derivatives having a mean particle diameter (d50) of from 0.25 to 5 μm and a coating with a silicon compound containing reactive groups.

Thus, according to the invention, the powdery filler consists of feldspar or feldspar derivatives. In particular, feldspar derivatives include materials deficient in silicon dioxide, so-called foids or feldspathoids.

The particles according to the invention have a mean particle diameter of from 0.25 to 5 μm. The mean particle diameter is referred to as d50. This means that 50% (by weight) of a particle mixture can pass a sieve of the corresponding diameter while 50% are retained.

The feldspar particles or feldspar derivative particles according to the invention have a coating with a silicon compound containing reactive groups. On the one hand, the coating must be capable of reacting with the filler, and on the other hand, reactive groups must remain. Such reagents are also employed in other fillers based on silica or glasses.

On the one hand, the reagents have a modified silicon compound capable of undergoing a reaction with the feldspar, for example, a trimethoxysilane group.

Further, the product preferably contains a polymerizable group, for example, an epoxide, an acrylate or methacrylate or a vinyl group, that is capable of polymerizing with a synthetic resin.

Reagents for this purpose are known to the skilled person. Typical reagents include, for example, γ-methacryloxypropyltrimethoxysilane.

In some embodiments, it is reasonable to mix different modifying reagents to coat the fillers.

As feldspars, members of the group of plagioclase feldspars or alkali feldspars have proven particularly suitable. Suitable minerals include, in particular, perthite, albite, oligoclase, andesine, labradorite, bytownite, anorthite as well as more SiO₂-deficient feldspar derivatives, such as nepheline, and mixtures thereof.

Preferably, the mean particle diameter of the feldspar is within a range of from 0.5 to 3.5 μm, preferably within a range of from 0.8 to 1.5 μm.

Preferably, the feldspar or feldspar derivative is transparent, for example, in order to enable photoinitiated polymerization in a system in which said feldspar or feldspar derivative is used as a filler.

Preferably, the light is a blue light and has a wavelength range of from 400 to 520 nm. Suitable light sources include halogen lamps or light-emitting diodes, so-called LEDs.

In one embodiment, the filler has an at least bimodal particle diameter distribution, i.e., there are two or more peaks in the grain size distribution. In such cases, preferably, one peak is within a range of from 0.5 to 1 μm, and the other peak is within a range of from 1 to 3.5 μm. Such bimodal or higher modal distributions are prepared, for example, by separately grinding and sieving materials to two grain size distributions of the desired size, followed by mixing them.

The mixing can be effected with equal weights of these grain groups or with different weights. For example, one grain size distribution could be employed in an amount of from 30 to 70% by weight, while the other is employed in a range of from 70 to 30% by weight.

In order to grind feldspar to a suitable size, in many cases, it is reasonable to employ two-step grinding.

A particularly preferred variant for the first grinding is so-called air jet autogenous grinding. In this method, particles are accelerated and forced to collide and ground thereby. Thus, feldspars can be ground in a grain size range down to about 1.5 μm.

For the further grinding, in particular, wet grinding methods are suitable, for example, using agitator ball mills. After the wet grinding methods, the filler is dried.

In a particularly preferred embodiment, grinding media are employed for grinding whose refractive index is close to the refractive index of the feldspar or feldspar derivative employed. Preferably, the difference in the refractive indices of the grinding media employed and the feldspar is not greater than 0.005. For example, in an agitator ball mill, glass beads of the corresponding refractive index may be employed as grinding media. Preferably, the ground material obtained contains less than 0.5% by weight of contaminations from grinding media wear particles; this can be determined, for example, by X-ray fluorescence analysis.

After drying, the filler is silanized in the known way. The methods are not basically different from the silanization of other supports.

In a particularly preferred embodiment, a dental composite material containing from 60 to 90% by weight of the powdery filler and from 10 to 40% by weight of a polymerizable resin is formed.

Preferably, the dental composite material is polymerized or cured by means of light. Usually, light having a wavelength range of from 400 to 520 nm is used.

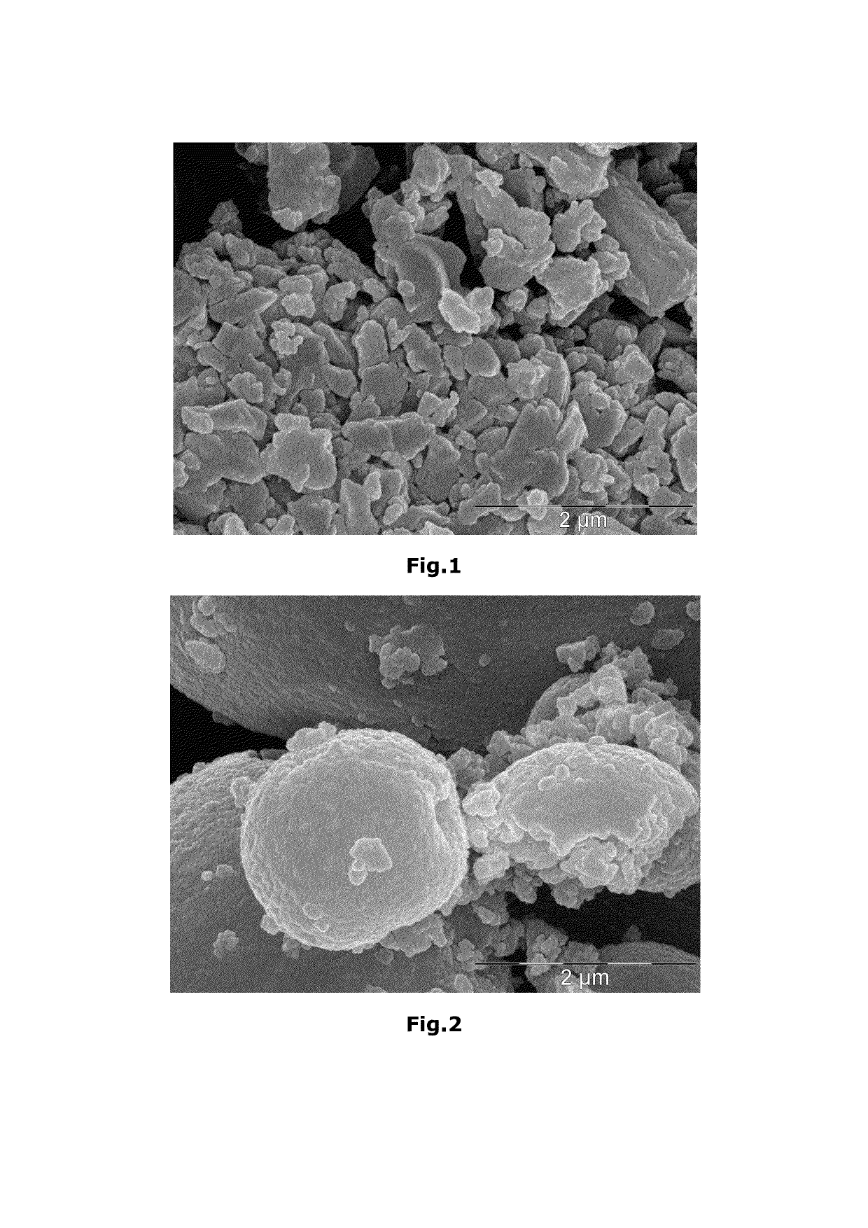

FIG. 1 shows a filler according to the invention in a grain size of 0.3 μm.

FIG. 2 shows the filler according to the invention in a grain size of 3.5 μm.

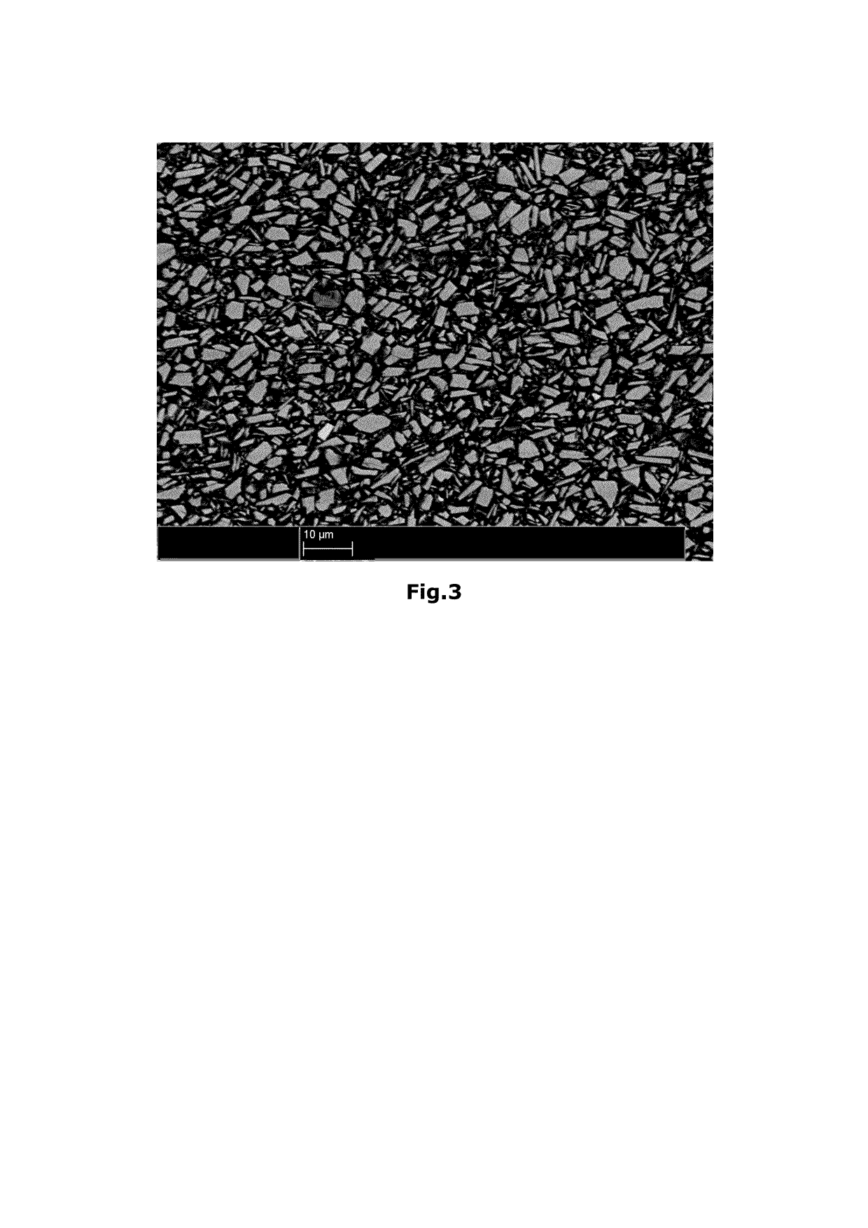

FIG. 3 shows a composite material obtained using the material according to the invention after curing and polishing the surface. The images are scanning electron micrographs.

### EXAMPLE 1

A polymerizable synthetic resin containing Bis-GMA (2,2-bis[4-(2-hydroxy-3-methylacryloxypropoxy)phenyl]propane together with TEGDMA (2-methyl-2-propenoic acid) was prepared. Camphorquinone and 2-dimethylaminoethyl methacrylate were employed as photoinitiators.

A feldspar coated with γ-methacryloxypropyltrimethoxysilane served as the feldspar. The mixing of the polymerizable resin and the filler was effected by manual mixing. The following feldspar grain sizes were used:
a) Grain size 0.3 μm
b) Grain size 0.8 μm
c) Grain size 3.5 μm
d) Mixture of fillers 0.8 μm and 3.5 μm in a weight ratio of 40:60

As Comparative Examples, there were employed:
C1: Barium glass, grain size 0.7 μm (GM 39923 of the company Schott)
C2: Barium glass, grain size 1.0 μm (GM 27884 of the company Schott)

### EXAMPLE 2

The following composite materials were prepared:
Filler a) 60%, synthetic resin 40%
Filler b) 67%, synthetic resin 33%
Filler c) 73%, synthetic resin 27%
Filler d) 74%, synthetic resin 36%
Filler C1 68%, synthetic resin 32%
Filler C2 72%, synthetic resin 28%

The curing was effected with a Dentacolor XS (Heraeus Kulzer) for 180 s for a 6 mm test specimen.

Subsequently, various properties of the materials were examined. The results are shown in the following Table.

| | Bending | Shear | Vickers | |
|---|---|---|---|---|
| | strength | strength | hardness | Roughness¹⁾ |
| | [MPa] | [MPa] | [HV 5-20] | Ra in [μm] |
| C1 0.7 μm | 115.6 | 22.6 | 47.0 | n.d. |
| C2 1.0 μm | 145.0 | 31.3 | 54.4 | n.d. |
| (a) 0.3 μm | 144.0 | 19.7 | 48.5 | 0.05 |
| (b) 0.8 μm | 212.0 | 29.7 | 53.9 | 0.05 |
| (c) 3.5 μm | 205.0 | 28.2 | 51.3 | 0.05 |
| (d) bimodal | 203.0 | 31.3 | 47.6 | 0.05 |

¹⁾after grinding with:
1st stage: roughening the surface with a carbide cutter
2nd stage: CompoMaster Coarse (Shofu)
3rd stage: CompoMaster (Shofu)
4th stage: DirectDia Paste; Super Snap Buff Disk (Shofu)
n.d.: not determined

As compared to usual dental filler materials based on strontium or barium glasses, the fillers according to the invention showed the same or in part improved mechanical properties. In the composite systems, very good curing results were achieved with the fillers according to the invention.

The linear shrinkage was from 1.4 to 1.7% and was thus better than in the prior art. High filler contents could be achieved, and nevertheless, a good workability of the composites according to the invention was found. The materials were highly transparent, so that they did not cause any change in color.

1. A powdery filler for dental materials comprising particles of feldspar or feldspar derivatives having a mean particle diameter (d50) of from 0.5 to 5 μm, said particles having a coating with a silicon compound containing reactive groups said dental materials being composite materials.

2. The powdery filler according to claim 1, wherein said reactive groups comprise polymerizable groups.

3. The powdery filler according to claim 2, wherein said polymerizable groups comprise epoxy or vinyl groups, preferably methacrylic or acrylic groups.

4. The powdery filler according to claim 1, wherein said feldspar is selected from the group of plagioclase feldspars or alkali feldspars.

5. The powdery filler according to claim 1, wherein said feldspar is selected from perthite, albite, oligoclase, andesine, labradorite, bytownite, anorthite as well as SiO₂-deficient feldspar derivatives, such as nepheline, and mixtures thereof.

6. The powdery filler according to claim 1, wherein said feldspar has a mean particle diameter (d50) of from 0.5 to 3.5 μm.

7. The powdery filler according to claim 1, wherein said feldspar is transparent.

8. The powdery filler according to claim 1, wherein said filler has a bimodal particle diameter distribution.

9. The powdery filler according to claim 8, wherein one peak of said bimodal distribution is within a range of from 0.5 to 1 μm, and a second peak is within a range of from 1 to 3.5 μm.

10. A process for preparing a powdery filler according to claim 1, with the following steps:
grinding feldspar
silanizing the particles with a reactive silicon compound.

11. A dental composite material containing
from 60 to 90% by weight of a powdery filler according to claim 1;
from 10 to 40% by weight of a polymerizable resin, wherein said polymerizable resin can react with the reactive groups.

12. The dental composite material according to claim 11, wherein said dental composite material can be cured by means of light.

13. A dental material containing a cured composite material according to claim 11.

14. Use of the powdery filler according to claim 1 as a filler in dental materials.

15. The powdery filler according to claim 1, wherein said feldspar has a mean particle diameter (d50) of from 0.8 to 1.5 μm.
